(19) United States
(12) Patent Application Publication
(10) Pub. No.: US 2024/0370482 A1
(43) Pub. Date: Nov. 7, 2024

(54) METHOD AND SYSTEM FOR IDENTIFYING ATTRIBUTE OF ENTITY

(71) Applicant: SAMSUNG SDS CO., LTD., Seoul (KR)

(72) Inventors: Su Yeon LEE, Seoul (KR); Ji Soo Lee, Seoul (KR); Hyo Young Kim, Seoul (KR)

(73) Assignee: SAMSUNG SDS CO., LTD., Seoul (KR)

(21) Appl. No.: 18/654,632

(22) Filed: May 3, 2024

(30) Foreign Application Priority Data
May 4, 2023 (KR) 10-2023-0058474

(51) Int. Cl. G06F 16/35; G16H 10/60

(52) U.S. Cl. CPC G06F 16/353; G16H 10/60

(57) ABSTRACT

A method is provided to identify attributes that are expressions within text that most accurately describe the meaning of a named entity included in the text. A method for entity attribute identifying in an embodiment of this disclosure may comprise recognizing one or more entities in an input text and selecting an attribute of a first entity included in the one or more entities among tokens included in the input text. The selecting of the attribute of the first entity may include selecting the attribute of the first entity among tokens that do not include the recognized one or more entities.

### CROSS-REFERENCE TO RELATED APPLICATION

This application claims priority from Korean Patent Application No. 10-2023-0058474 filed on May 4, 2023 in the Korean Intellectual Property Office, and all the benefits accruing therefrom under 35 U.S.C. 119, the contents of which in its entirety are herein incorporated by reference.

### BACKGROUND

### 1. Technical Field

The present disclosure relates to a method and system for identifying an attribute of an entity. More particularly, the present disclosure relates to a method and system for identifying a description presenting information most related to the meaning of an entity included in a text in the text.

### 2. Description of the Related Art

A named entity recognition (NER) technology is provided. When the named entity recognition technology is used, it is possible to recognize named entities in an unstructured text and classify types of the recognized named entities. The types of the named entities have been defined through several standards or the like. For example, TTAK.KO-10.0852 (Tag Set and Tagged Corpus for Named Entity Recognition, Telecommunications Technology Association Standard (TTAS)) defines 15 types of named entities, and Definition of Korean Named-Entity Task and Cover Page Standardization Technical Report and entities morpheme corpus produced based on Definition of Korean Named-Entity Task and Cover Page Standardization Technical Report (https://github.com/kmounlp/NER) defines 10 types of named entities.

An aim of technological advancement of the named entity recognition technology is to accurately extract specific types of entities. That is, it is difficult to identify the meaning of a specific entity in an unstructured text using only the named entity recognition technology.

The following example sentence, “A Electronics is currently still owning a 19.9% stake in B Electronics, and as of 2013, this company had assets of 191.2 billion won and liabilities of 182.5 billion won, which means its assets are greater than its liabilities, but has run a deficit of 64.2 billion won.” includes a money type of multiple entities. When unmanned task automation is to be performed on such an example sentence, it will be necessary to grasp the meaning of each of the money type of entities. For example, it will be necessary to be able to grasp whether the 191.2 billion won is assets, liabilities, or a deficit. However, the named entity recognition technology aiming to recognize the named entities and classifying types of the recognized named entities may not grasp the meaning of the entities.

Meanwhile, a technology called relation extraction (RE) is provided. The relation extraction is a task well known together with the named entity recognition in natural language processing. The relation extraction is a task that derives a relation between two extracted entities, and is a task that mainly focuses on deriving a relation between LOC (location name)-ORG (organization)-PER (person). In other words, the relation extraction derives a relation between two entities under the assumption that the two entities have been extracted. For example, the relation extraction mainly aims to derive relations between PER-ORG-LOC corresponding to named entities such as top members or employees of an organization (org:top_members/employees, ORG-PER), and per:sibling (sibling relation, PER-PER), members of the organization (org:member_of).

Most relation extraction tasks aim to build general knowledge through identification of the relation between the entities. It is necessary to be able to identify relations between various entities in order to build the general knowledge, and thus, relation extraction tasks so far aim to identify multiple relation classes. When KLUE-RE (https://klue-benchmark.com/tasks/70/data/download), which is Korean relation extraction (RE) data, is confirmed, among 31 relation classes, no-relation occupies 29.4%, org:top_members/employees occupies 13.2%, per:employee_of occupies 11.0%, and per:title occupies 6.5%.

In addition, as shown in Table 1, when Korean RE (klue-RE, https://klue-benchmark.com/) data are confirmed, relations between ORG-PER-LOC occupies most (82.7%) of the relation classes. In this case, relations related to @NOH are a total of 103 cases, which are very small, corresponding 0.45% of meaningful relations (22,936 cases) excluding no-relation.

TABLE 1

| | | # of | # of samples |
|---|---|---|---|
| Subject | Object | samples | (exclude |
| entity_type | entity_type | (total) | no_relation) |
| DAT | ORG | 2,110 | 528 |
| DAT | PER | 2,139 | 1,634 |
| LOC | ORG | 1,776 | 1,228 |
| LOC | PER | 1,785 | 1,475 |
| NOH | ORG | 260 | 68 |
| NOH | PER | 153 | 35 |
| ORG | ORG | 5,100 | 3,142 |
| ORG | PER | 4,246 | 3,505 |
| PER | ORG | 4,779 | 4,378 |
| PER | PER | 5,009 | 3,610 |
| POH | ORG | 1,659 | 934 |
| POH | PER | 3,454 | 2,399 |

That is, an existing relation extraction technology aims to build general knowledge, and thus, there are many kinds of relation classes and there is a limitation that it is impossible to extract relations between types of entities important for task automation.

In addition, an existing relation extraction task may not identify relations between entities well for bullet-type expressions or noun enumeration-type expressions rather than a document written in descriptive expressions. Considering that some of texts, which are processing targets for the task automation, are bullet-type expressions or simple noun enumeration-type expressions, the existing relation extraction task capable of extracting relations between entities only in a document written in descriptive expressions, could not provide a sufficient function for the task automation.

In conclusion, when a system that understands the meaning of entities included in a text for the task automation is to be implemented, it is difficult to implement a function of accurately identifying other descriptions of a text accurately describing a type of entity meaningful for the task automation only by utilizing an existing named entity recognition technology and relation extraction technology.

### SUMMARY

Aspects of the present disclosure provide a method and system for identifying a description presenting information closest to the meaning of an entity included in an unstructured text in the unstructured text.

Aspects of the present disclosure also provide a method and system for performing task automation by finding the meaning of an entity included in an unstructured task-related text from the unstructured task-related text.

Aspects of the present disclosure also provide a method and system for identifying a description presenting information closest to the meaning of an entity, not only for a text comprising descriptive sentences, but also for a text comprising bullet-type expressions or noun enumeration forms.

Aspects of the present disclosure also provide a method and system for generating a natural language processing-related training dataset including a text, a target entity, which is any one of entities of the text, and a description presenting information closest to the meaning of the target entity.

However, aspects of the present disclosure are not restricted to those set forth herein. The above and other aspects of the present disclosure will become more apparent to one of ordinary skill in the art to which the present disclosure pertains by referencing the detailed description of the present disclosure given below.

According to an aspect of the present disclosure, there is provided a method for identifying an attribute of an entity, the method being performed by a computing system. The method may comprise recognizing one or more entities in an input text and selecting an attribute of a first entity included in the one or more entities among tokens included in the input text. The selecting of the attribute of the first entity may include selecting the attribute of the first entity among tokens that do not include the recognized one or more entities.

The recognizing of the one or more entities may include recognizing only any one type of entity of a plurality of predetermined types in the input text. Further the recognizing of only any one type of entity of the plurality of predetermined types may include recognizing a quantity type of entity or a code type of entity, and the method may further comprise performing a robotic process automation (RPA) task using the first entity and the attribute of the first entity. Still further, the recognizing of only any one type of entity of the plurality of predetermined types may include recognizing a quantity type of entity or a code type of entity, and the method may further comprise retrieving an input field corresponding to the attribute of the first entity and inputting the first entity as a value of the retrieved input field. The input text is a natural language text may be included in a medical record, and the input field is included in one of a plurality of input may form belonging to an electronic medical record (EMR).

The method may further comprise generating training data, the training data comprising entity-attribute pairs, each entity-attribute pair including a corresponding entity of the one or more entities and an attribute of the corresponding entity.

The selecting of the attribute of the first entity among the tokens that do not include the recognized one or more entities may include segmenting the input text into a plurality of unit texts; and selecting the attribute of the first entity among tokens that is included in a unit text including the first entity and do not include the recognized one or more entities. The selecting of the attribute of the first entity among the tokens that is included in the unit text may include the first entity and do not include the recognized one or more entities includes skipping the selecting of the attribute for a unit text in which any one type of named entity of a plurality of predetermined types is not recognized among the plurality of unit texts.

The selecting of the attribute of the first entity may include selecting a plurality of candidate attributes among the tokens that do not include the recognized one or more entities, using a part of speech of each token, determining a relation class between each of the plurality of candidate attributes and the first entity and selecting the attribute of the first entity using the determined relation class. The determining of the relation class may include determining the relation class as any one of three classes of relations: is-a, part-of, and no-relation. The determining of the relation class may include determining a plurality of relation classes corresponding to a type of the first entity and determining the relation class between each of the plurality of candidate attributes and the first entity as any one of the determined relation classes. The determining of the plurality of relation classes corresponding to the type of the first entity may include determining at least one of two relation classes: is-a and part-of as some of the plurality of relation classes corresponding to the type of the first entity and determining a class: no-relation as the other of the plurality of relation classes corresponding to the type of the first entity. The determining the relation class between each of the plurality of candidate attributes and the first entity may include selecting a candidate attribute having a relation class corresponding to a type of the entity as the attribute of the entity.

The determining of the relation class may include determining the relation class between each of the plurality of candidate attributes and the first entity using a first relation extraction model when a type of the first entity is a first type and determining the relation class between each of the plurality of candidate attributes and the first entity using a second relation extraction model different from the first relation extraction model when the type of the first entity is a second type different from the first type. The first relation extraction model and the second relation extraction model may be models trained based on machine learning, receiving input data including a sentence, an entity, and an attribute, and outputting data related to what a relation between the entity and the attribute belongs to any one of a plurality of relation classes. The first relation extraction model may output data related to what the relation between the entity and the attribute belongs to any one of a plurality of first relation classes. The second relation extraction model outputs data related to what the relation between the entity and the attribute may belong to any one of a plurality of second relation classes, and at least one of the plurality of first relation classes may include one or more first non-common relation classes which is not included in the plurality of second relation classes and at least one of the plurality of second relation classes may include one or more second non-common relation classes which is not included in the plurality of first relation classes.

The selecting of the plurality of candidate attributes may include excluding some of the plurality of candidate attributes from the plurality of candidate attributes using a relation between each of the plurality of candidate attributes and the entity, and the selecting of the attribute of the first entity using the determined relation class may include determining a token distance between a candidate attribute and the entity for each of candidate attributes remaining after the excluding of some of the plurality of candidate attributes; and selecting the attribute of the first entity among the candidate attributes remaining after the excluding of some of the plurality of candidate attributes, using the token distance of each of the candidate attributes. The selecting of the attribute of the first entity among the candidate attributes remaining after the excluding of some of the plurality of candidate attributes, using the token distance of each of the candidate attributes may include determining a context distance between the candidate attribute and the first entity; and selecting the attribute of the first entity among the candidate attributes remaining after the excluding of some of the plurality of candidate attributes, using the token distance and the context distance of each of the candidate attributes. The determining of the context distance and the selecting of the attribute of the first entity among the candidate attributes remaining after the excluding of some of the plurality of candidate attributes, using the token distance and the context distance of each of the candidate attributes may be performed only when the input text is a descriptive sentence.

The selecting of the attribute of the first entity may include selecting a plurality of candidate attributes among the tokens that do not include the recognized one or more entities, using a part of speech of each token, determining a token distance between each of the plurality of candidate attributes and the first entity; and selecting the attribute of the first entity by partially using the token distance of each of the candidate attributes. The selecting of the attribute of the first entity may further include determining a relation class between each of the plurality of candidate attributes and the first entity, and the selecting of the attribute of the first entity by partially using the token distance of each of the candidate attributes may include selecting the attribute of the first entity using the token distance of each of the candidate attributes and the determined relation class.

The selecting of the attribute of the first entity may include selecting a plurality of candidate attributes among the tokens that do not include the recognized one or more entities, using a part of speech of each token, retrieving a record for each of the plurality of candidate attributes from a pre-stored statistical table and selecting the attribute of the first entity using the retrieved record of each of the plurality of candidate attributes. The pre-stored statistical table may include a record of each attribute. The record may include a type of an entity, the number of times of extraction of the entity, information on a distance between an attribute and the entity, and a confidence score, and the retrieved record may be a record of a candidate attribute having a type of an entity coinciding with a type of the first entity.

The selecting of the attribute of the first entity using the retrieved record of each of the plurality of candidate attributes may include calculating a difference between a record of the retrieved record of each of the plurality of candidate attributes and a distance between the first entity and a first candidate attribute on the input text, adjusting the confidence score of the retrieved record for each of the plurality of candidate attributes using the calculated difference of each of the plurality of candidate attributes, and selecting the attribute of the first entity using the adjusted confidence score of each of the plurality of candidate attributes. The selecting of the attribute of the first entity further may include determining a relation class between each of the plurality of candidate attributes and the first entity, and the selecting of the attribute of the first entity by partially using the token distance of each of the candidate attributes may include selecting the attribute of the first entity using the token distance of each of the candidate attributes and the determined relation class.

The selecting of the attribute of the first entity may include selecting a plurality of candidate attributes among the tokens that do not include the recognized one or more entities, using a part of speech of each token, retrieving a record for each of the plurality of candidate attributes from a pre-stored statistical table and selecting the attribute of the first entity using the retrieved record of each of the plurality of candidate attributes. The pre-stored statistical table may include a record of each attribute, the record may include a type of an entity, the number of times of extraction of the entity, information on a distance between an attribute and the entity, and a confidence score, and the retrieved record may be a record of a candidate attribute having a type of an entity coinciding with a type of the first entity.

The selecting of the attribute of the first entity using the retrieved record of each of the plurality of candidate attributes may include calculating a difference between a record of the retrieved record of each of the plurality of candidate attributes and a distance between the first entity and a first candidate attribute on the input text, adjusting the confidence score of the retrieved record for each of the plurality of candidate attributes using the calculated difference of each of the plurality of candidate attributes and selecting the attribute of the first entity using the adjusted confidence score of each of the plurality of candidate attributes. The record further may include information on a relation class between the attribute and the entity, and the retrieved record may be a record of the candidate attribute having a relation class value coinciding with a relation class between the first entity and the candidate attribute.

The selecting of the attribute of the first entity using the retrieved record of each of the plurality of candidate attributes may include calculating a confidence score of a first candidate attribute using a token distance between the first candidate attribute and the first entity and a relation class between the first candidate attribute and the first entity when a record corresponding to the first candidate attribute of the plurality of candidate attributes is not retrieved from the pre-stored statistical table and selecting the attribute of the first entity by comparing the calculated confidence score with a confidence score of the retrieved record.

The selecting of the attribute of the first entity among the tokens that do not include the recognized one or more entities may include selecting a plurality of candidate attributes among the tokens that do not include the recognized one or more entities, using a part of speech of each token, constructing input data for each of the plurality of candidate attributes, the input data including a target text, the first entity, a candidate attribute, and a relation class between the first entity and the candidate attribute and inputting the input data for each of the plurality of candidate attributes into a pre-trained deep learning-based attribute identification model and selecting the attribute of the first entity among the plurality of candidate attributes using data output from the pre-trained deep learning-based attribute identification model. The pre-trained deep learning-based attribute identification model may be generated through additional training using training data comprising the target text, an entity, an attribute, a relation class between the entity and the attribute, based on a deep learning-based base model performing a relation extraction (RE) task between the entities.

According to other aspect of the present disclosure, there is provided a system for identifying an attribute of an entity. The system may comprise a storage, a communication interface, a memory configured to load a computer program and one or more processors configured to execute the computer program. The computer program may include an instruction configured to cause the one or more processors to recognize one or more entities in an input text received through the communication interface or stored in the storage; and an instruction configured to cause the one or more processors to select an attribute of a first entity included in the one or more entities among tokens included in the input text, and the instruction configured to cause the one or more processors to select the attribute of the first entity includes an instruction configured to cause the one or more processors to select the attribute of the first entity among tokens that do not include the recognized one or more entities.

### BRIEF DESCRIPTION OF THE DRAWINGS

The above and other aspects and features of the present disclosure will become more apparent by describing in detail exemplary embodiments thereof with reference to the attached drawings, in which:

FIGS. 1 to 3 are block diagrams illustrating configurations of a processing system according to an exemplary embodiment of the present disclosure;

FIG. 4 is a flowchart of a method for identifying an attribute of an entity according to another exemplary embodiment of the present disclosure;

FIG. 5 is a detailed flowchart for describing some operations of the method for identifying an attribute of an entity described with reference to FIG. 4;

FIG. 6 is a flowchart of a method for identifying an attribute of an entity according to still another exemplary embodiment of the present disclosure;

FIG. 7 is a diagram for describing entity type division in any one of existing named entity recognition technologies;

FIG. 8 is a diagram for describing a named entity recognition result for an example text;

FIG. 9 is a diagram for describing a relation class classified as a relation between a target entity and a candidate attribute in some exemplary embodiments of the present disclosure;

FIG. 10 is a diagram for describing that a classified relation class changes depending on a type of a target entity varies in some exemplary embodiments of the present disclosure and an entity type that is a target of attribute identification in some exemplary embodiments of the present disclosure;

FIG. 11 is a diagram for describing a named entity recognition result according to some exemplary embodiments of the present disclosure for the example text of FIG. 8;

FIG. 12 is a diagram for describing an attribute identification process according to some exemplary embodiments of the present disclosure according to the named entity recognition result of FIG. 11;

FIG. 13 is a diagram for describing calculation of a distance between an entity and a candidate attribute, which is some operations of the method for identifying an attribute of an entity described with reference to FIG. 4;

FIG. 14 is a diagram for describing a result of performing entity attribute identification on the example text of FIG. 8 according to some exemplary embodiments of the present disclosure;

FIGS. 15 to 17 are diagrams for describing a statistical table referenced in some exemplary embodiments of the present disclosure;

FIG. 18 is a diagram for describing human filtering for an entity attribute identification result that may be performed in some exemplary embodiments of the present disclosure;

FIG. 19 is a diagram for describing an example format of training data for a deep learning-based model of entity attribute identification that may be performed in some exemplary embodiments of the present disclosure; and

FIG. 20 is a block diagram illustrating a hardware configuration of a computing system described in some exemplary embodiments of the present disclosure.

### DETAILED DESCRIPTION

Hereinafter, example embodiments of the present disclosure will be described with reference to the attached drawings. The advantages and features of the present disclosure and methods of accomplishing the same may be understood more readily by reference to the following detailed description of example embodiments and the accompanying drawings. The present disclosure may, however, be embodied in many different forms and should not be construed as being limited to the embodiments set forth herein. Rather, these embodiments are provided so that this disclosure will be thorough and complete and will fully convey the concept of the disclosure to those skilled in the art, and the present disclosure will be defined by the appended claims and their equivalents. In describing the present disclosure, if it is determined that a detailed description of a related known configuration or function may obscure the gist of the present invention, the detailed description will be omitted.

First, some terms mentioned in the present disclosure will be described.

### [Entity]

An entity refers to an expression having a specific meaning in a sentence or a document, such as a person's name, an organization name, or a location name. However, it is to be noted that the entity is not limited to a proper noun such as the person's name, the organization name, or the location name described above, and is a concept also including an expression for a numerical value expressing a specific meaning. The entity may be extracted through a named entity recognition (NER) technology for recognizing an entity in an input text.

### [Attribute]

An attribute is a description evaluated to most appropriately express the meaning of the entity described above, and is selected among tokens described in the input text. The token may be obtained as a result of inputting the input text into a tokenization performing module such as a morphological analyzer.

### 1. Processing System

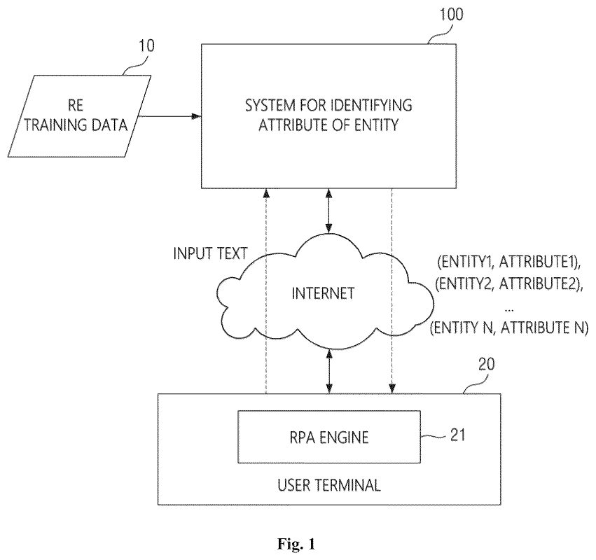

Hereinafter, configurations and operations of a processing system according to an exemplary embodiment of the present disclosure will be described with reference to FIGS. 1 to 3. FIGS. 1 to 3 illustrate configuration examples of different forms of a processing system according to the present exemplary embodiment. The processing system according to the present exemplary embodiment may be understood as a system that performs an analysis operation on an unstructured text in a natural language form.

The processing system according to the present exemplary embodiment may include a system 100 for identifying an attribute of an entity. The system 100 for identifying an attribute of an entity may comprise one or more computing devices. For example, the system 100 for identifying an attribute of an entity may comprise one or more cloud compute instances. That is, the system 100 for identifying an attribute of an entity may comprise at least some compute instances of one or more virtual machines and one or more containers.

In addition, the system 100 for identifying an attribute of an entity may be configured to include both an on-premise physical server and the cloud compute instances. For example, in consideration of a situation where a text with high security requirements should be processed, a module analyzing an input text or at least temporarily storing the input text is implemented on an on-premise physical server positioned in an internal network blocked from the Internet by a firewall, and other modules may be configured using the cloud compute instances.

The system 100 for identifying an attribute of an entity may include a named entity recognizer (not illustrated) performing named entity recognition, a tokenizer (not illustrated) performing morphological analysis, and a relation extractor (not illustrated) determining a relation class between each entity and a candidate attribute. The tokenizer may be implemented using an existing well known open source morpheme analyzer or the like. For example, see Mecab (https://github.com/taku910/mecab) as an example of the open source morphological analyzer. The tokenizer may also be implemented by executing additional training using the open source morphological analyzer as a base model so that it may be specialized for a language or a domain of the input text.

The system 100 for identifying an attribute of an entity may provide an attribute identification function only for a type of entity including important information in terms of task automation. That is, the named entity recognizer may include a named entity recognition model machine-learned so as to recognize only any one type of entity of a plurality of predetermined types.

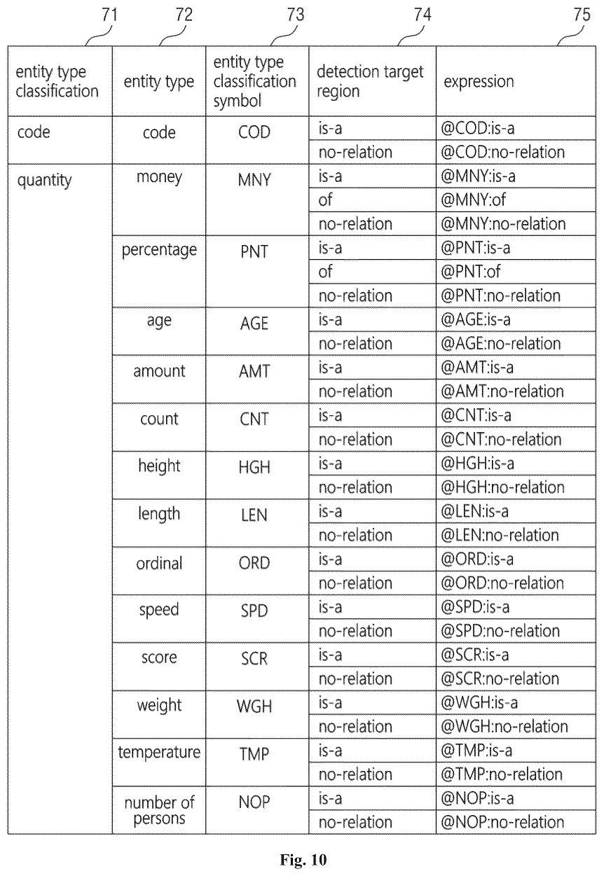

The predetermined type may be a quantity type of entity or a code type of entity, which is an important type of entity in the task automation. Examples of each of the quantity type of entity and the code type of entity will be described later with reference to FIG. 10.

The system 100 for identifying an attribute of an entity selects only non-entity tokens that do not include the recognized entity among the tokens included in the input text as a candidate attribute. That is, the system 100 for identifying an attribute of an entity provides a new method for selecting an attribute of an entity among the non-entity tokens by improving the fact that relations between entities that were attended in an existing relation extraction task may not provide meaningful information in terms of the task automation.

In other words, the relation extractor performs an operation of determining a relation class between a plurality of non-entity tokens and an entity unlike the existing relation extraction task that determines a relation class between entities. That is, it may be understand that the existing relation extraction task generates data of (sentence, subject entity, object entity, relation class), while the relation extractor generates data of (sentence, target entity, candidate attribute, relation class).

Here, it may be understood that the target entity is a type of entity including important information in terms of the task automation, that is, any one type of entity of a plurality of predetermined types. As described above, at least some of the non-entity tokens may be the candidate attribute.

Here, the non-entity token may refer to all tokens other than a token including a type of entity that may be recognized by the named entity recognizer, that is, any one type of entity of the plurality of predetermined types. That is, the system 100 for identifying an attribute of an entity may select the attribute among types of entities other than the plurality of predetermined types and pure non-entity tokens.

In addition, in some other exemplary embodiments, the non-entity token may refer to all tokens other than tokens including entities in a broad sense. The entities in the broad sense are not limited to the plurality of predetermined types of entities, but may include all of various types of entities recognized by existing named entity recognizers. In other words, the named entity recognizer may select the attribute only among the pure non-entity tokens.

Considering that the number of entities included in the text is smaller than the number of non-entity tokens, an amount of calculation of the relation extractor may be increased compared to the existing relation extraction task that determines the relation class between the entities. In addition, the purpose of determining the relation class by the system 100 for identifying an attribute of an entity is to accurately identify the attribute of the entity through a relation between the entity and the candidate attribute rather than to know the relation between the entity and the candidate attribute. In consideration of this, the relation extractor is trained to be able to classifying only a smaller number of relation classes than the existing relation extraction task and may thus reduce an overall amount of calculation.

The “smaller number of relation classes” are three classes: is-a, part-of, and no-relation or two classes: is-a and no-relation. The relation extractor may classify the relation class into any one of the three classes described above regardless of a type of the entity or may classify the relation class into any one of the two classes described above regardless of the type of the entity. In addition, the relation extractor may classify the relation class into any one of the three classes described above or the two classes described above depending on the type of the entity. This will be described in detail later with reference to FIGS. 9 and 10.

The relation extractor may use a relation extraction model trained by at least partially using relation extraction (RE) training data 10 for machine learning of a machine learning-based model that performs the existing relation extraction task. As an example of the RE training data 10, training data (https://klue-benchmark.com/tasks/70/data/download) of a relation extraction task of Korean Language Understanding Evaluation (KLUE) (http://klue-benchmark.com) may be utilized.

In addition, in another exemplary embodiment, the relation extractor may use a relation extraction model generated through additional training that uses additional training data, using an existing relation extraction model generating the data of (sentence, subject entity, object entity, relation class) according to the existing relation extraction task as a base model. In this case, the relation extraction model generated through the additional training will include a new classifier instead of a classifier of the base model or include a new classifier additionally connected to the classifier of the base model. The new classifier will classify the relation class into any one of the three classes described above or the two classes described above. The additional training data may comprise (sentence, entity, attribute, relation class).

Hereinafter, the processing system according to the present exemplary embodiment will be additionally described, focusing on an operation of the system 100 for identifying an attribute of an entity and a connection relation between other components of the processing system.

As illustrated in FIG. 1, the system 100 for identifying an attribute of an entity may receive an input text from a user terminal 20. The user terminal 20 may encrypt the input text and transmit the encrypted input text to the system 100 for identifying an attribute of an entity. The system 100 for identifying an attribute of an entity may analyze the input text and transmit attribute information for each entity included in the input text as a result of the analysis to the user terminal 20.

The system 100 for identifying an attribute of an entity may recognize one or more entities in a named entity recognition module, and select an attribute of each entity among non-entity tokens included in the input text.

As described above, the system 100 for identifying an attribute of an entity may be a system specialized for processing an unstructured natural language text for the task automation. For example, the system 100 for identifying an attribute of an entity may be involved in a process in which an RPA bot is executed using a robotic process automation (RPA) technology. For example, when an RPA engine 21 installed in the user terminal 20 executes the RPA bot including an action of finding a value of a specific attribute for an email body, a document file, or a web document, the RPA engine 21 may extract the input text included in the email body, the document file, or the web document and transmit the input text to the system 100 for identifying an attribute of an entity. The RPA engine 21 will receive data of (entity, attribute) as a response to the transmission of the input text from the system 100 for identifying an attribute of an entity, and will recognize an entity including the value of the specific attribute for performing the action.

In some other exemplary embodiments, a function of the system 100 for identifying an attribute of an entity may be implemented in the RPA engine 21.

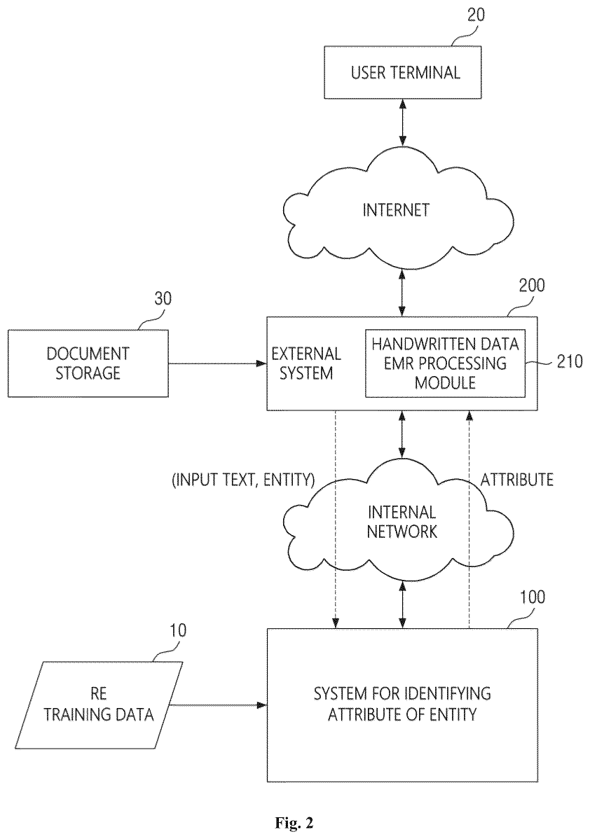

As illustrated in FIG. 2, the processing system according to the present exemplary embodiment may include a system 100 for identifying an attribute of an entity and an external system 200. The system 100 for identifying an attribute of an entity may provide an entity attribute identification function to the external system 200 that provides a service to the user terminal 20. The external system 200 may include a multipurpose system that processes an unstructured natural language text stored in a document storage 30. As an example, a handwritten data electronic medial record (EMR) processing module 210 of a system that understands various unstructured natural language texts input in a medical treatment process and automatically builds EMR data using an understanding result may be included in the external system 200.

Due to the urgency of a medical treatment situation, a natural language text input in the medical treatment process has many simple enumerated noun forms.

Conventionally, a person should directly understand such a natural language text and input EMR data according to an understanding result, which is inefficient. The system illustrated in FIG. 2 may recognize a quantity type of entity such as various examined numerical values or a code type of entity such as disease classification codes, retrieve an input field of the EMR data corresponding to an attribute of the recognized entity, and automatically input the recognized entity as a value of the retrieved input field. It may be understood that the input field is included in any one of a plurality of input forms belonging to an EMR.

In some exemplary embodiments, the external system 200 may include the named entity recognizer. That is, the external system 200 may recognize the entity included in the input text by selecting the input text among texts stored in the document storage 30 and inputting the input text into the named entity recognizer. In this case, the external system 200 may select an entity of interest whose attribute need to be grasped, transmit an attribute request including the input text and the entity of interest to the system 100 for identifying an attribute of an entity, and receive an attribute of the entity of interest included in the attribute request as a response to the attribute request.

In some exemplary embodiments, the external system 200 may include the RPA engine described with reference to FIG. 1.

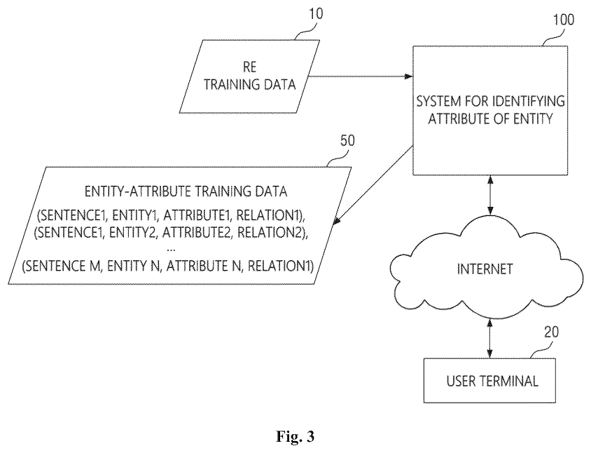

As illustrated in FIG. 3, the processing system according to the present exemplary embodiment may generate entity-attribute training data. In this case, the system 100 for identifying an attribute of an entity included in the processing system according to the present exemplary embodiment may receive a training data generation command from the user terminal 20 and generate the entity-attribute training data. The training data generation command may include a path of a target text.

The system 100 for identifying an attribute of an entity may perform an operation of receiving the target text, segmenting the target text into a plurality of input texts, recognizing one or more entities in each of the plurality of input texts, and selecting an attribute corresponding to each recognized entity among non-entity tokens included in the input text. As a result, the system 100 for identifying an attribute of an entity may generate a set of training data including (sentence including entity, entity, attribute).

As described above, the system 100 for identifying an attribute of an entity may identify a type of the entity in an attribute identification process. As a result, the system 100 for identifying an attribute of an entity may identify a set of training data including (sentence including entity, entity, type of entity, attribute)

As described above, the system 100 for identifying an attribute of an entity may determine a relation class between the entity and the attribute in the attribute identification process, and may generate a set of training data in which such relation class information is reflected. That is, the system 100 for identifying an attribute of an entity may generate a set of training data including (sentence including entity, entity, relation class between entity and attribute, and attribute) or generate a set of training data including (sentence including entity, entity, type of entity, relation class between entity and attribute, attribute).

Hereinabove, the configurations and the operations of the processing system according to an exemplary embodiment of the present disclosure have been described with reference to FIGS. 1 to 3. An operating method of the system 100 for identifying an attribute of an entity of the processing system according to the present disclosure may be understood in more detail with reference to other exemplary embodiments to be described later. In addition, a technical idea that may be understood through the above-described exemplary embodiments of the processing system according to the present exemplary embodiment may be reflected in other exemplary embodiments to be described later even though not specified separately.

### 2. Method for Identifying Attribute of Entity

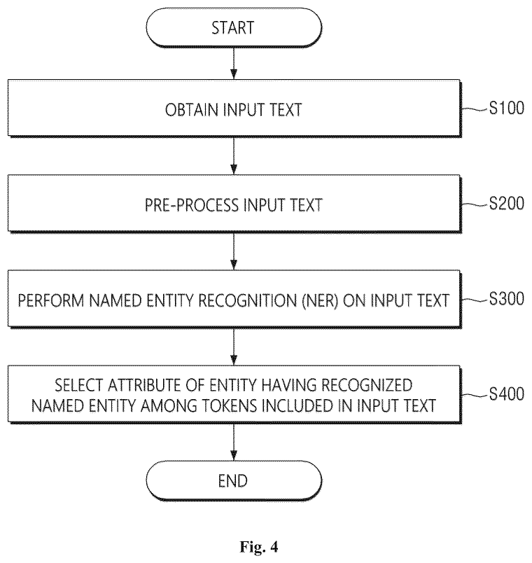

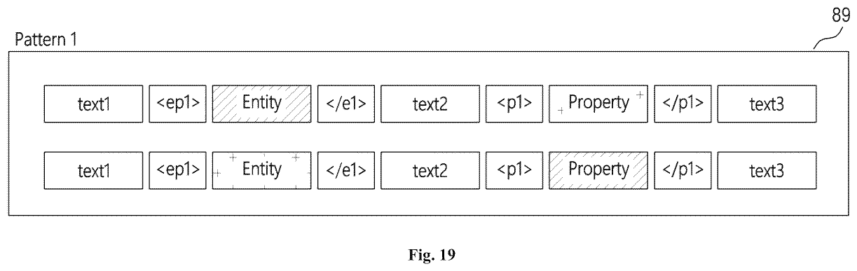

A method for identifying an attribute of an entity according to another exemplary embodiment of the present disclosure will be described with reference to FIGS. 4 to 19. The method for identifying an attribute of an entity according to the present exemplary embodiment may be performed by one or more computing systems. In addition, some operations of the method for identifying an attribute of an entity according to the present exemplary embodiment may be performed by a first computing device, and the other operations of the method for identifying an attribute of an entity according to the present exemplary embodiment may be performed by a second computing device. For example, some operations of the method for identifying an attribute of an entity according to the present exemplary embodiment may be performed by an on-premise physical server, and the other operations of the method for identifying an attribute of an entity according to the present exemplary embodiment may be performed by a cloud compute instance. Hereinafter, it will be understood that when a subject performing each operation is omitted, the subject is the computing system.

FIG. 4 is a flowchart illustrating general operations of a method for identifying an attribute of an entity according to the present exemplary embodiment. As illustrated in FIG. 4, the method for identifying an attribute of an entity according to the present exemplary embodiment may include obtaining an input text (S100), pre-processing the input text (S200), performing named entity recognition (NER) on the preprocessed input text (S300), and selecting an attribute of an entity having a recognized named entity (S400).

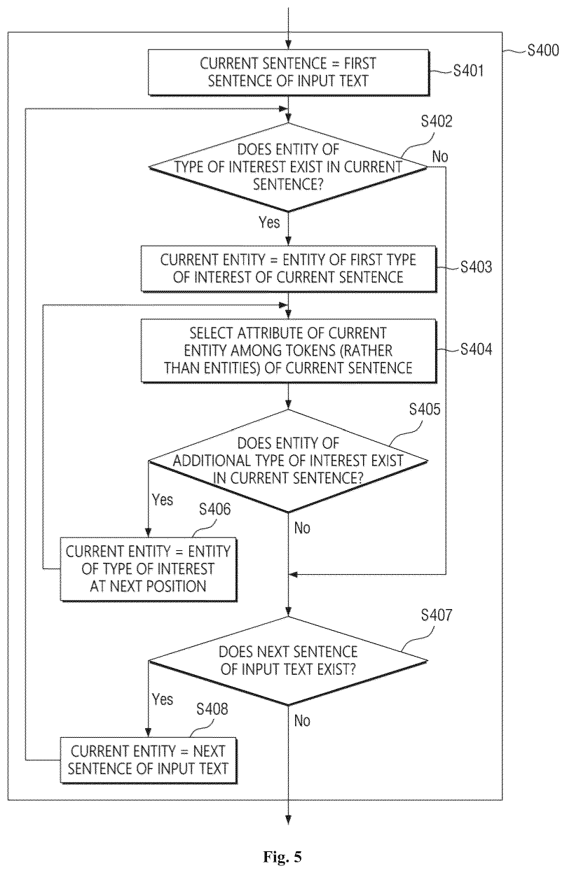

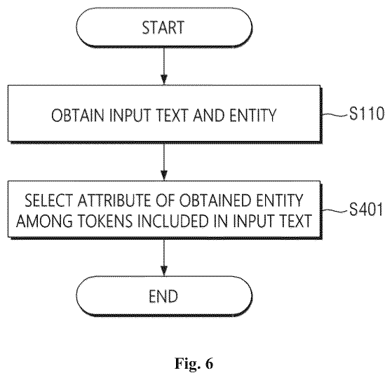

In addition, FIG. 6 is another flowchart illustrating general operations of a method for identifying an attribute of an entity according to the present exemplary embodiment. FIG. 6 is a flowchart illustrating a method for identifying an attribute of an entity in a form in which an input text and an entity, which is an attribute selection target, are designated by a user terminal or the like, and the method for identifying an attribute of an entity according to the present exemplary embodiment may include obtaining an input text and information on an attribute selection target entity (S110) and selecting an attribute of the obtained entity among tokens included in the input text (S401). It may be understood that S400 is different from S401 only in that S400 selects attributes of respective entities included in the input text, while S401 selects an attribute of only the entity obtained in S110 among the respective entities included in the input text, and other operations are same as each other. S400 and S401 will be described later with reference to FIG. 5.

Hereinafter, respective operations will be described with reference to FIG. 4.

The obtaining of the input text (S100) may include an operation of performing the input text from the external device or may include an operation of loading the input text from a pre-stored file or database.

Through the pre-processing of the input text (S200), in some exemplary embodiments, the input text may be segmented into a plurality of unit texts For example, each of the unit texts may be segmented based on a predetermined delimiter such as a period, a comma, a line break, two or more spaces, or a semicolon. For example, when the delimiter is the period, each of the unit texts will be a sentence. The reason why the delimiter is not limited to the period as described above is to identify an attribute of an entity even for an input text that is not expressed in the form of a descriptive sentence and is expressed in the form of a bullet-type expression or a simple word enumeration.

The reason why the segmentation into the unit text is performed is to narrow a search scope for an attribute of an entity. That is, the attribute of an entity recognized in the unit text may be searched for only within the unit text.

In addition, an expression may be corrected through the preprocessing of the input text (S200). For example, an English abbreviation expression may be replaced with a corresponding original text expression, a symbol may be replaced with a text expression corresponding to the symbol, and when a replacement target expression included in an automatic replacement dictionary is found, the replacement target expression may be replaced with a post-replacement expression described in the automatic replacement dictionary.

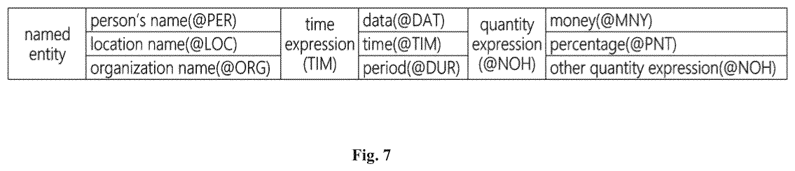

By performing the named entity recognition (NER) on the preprocessed input text (S300), one or more entities included in the input text may be recognized. In this case, as described above, in some exemplary embodiments, a target of the named entity recognition may be limited to a plurality of predetermined types of entities. Unlike existing named entity recognition in which entity types related to named entities such as a person's name (@PER), a location name (@LOC), and an organization name (@ORG) are also recognition targets, as illustrated in FIG. 7, in the named entity recognition according to some exemplary embodiments of the present disclosure, as illustrated in FIG. 10, only a predetermined type of entities classified (71) into a code type of entity and a quantity type of entity may be recognized.

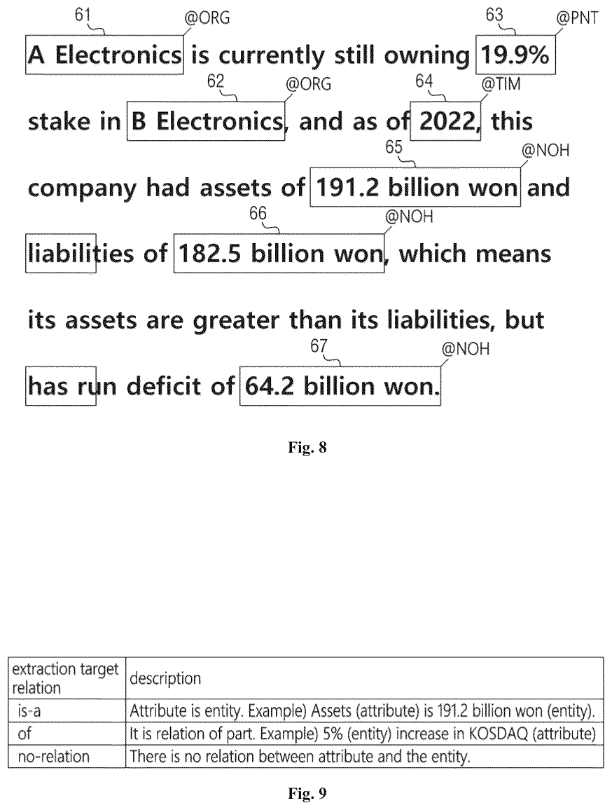

FIG. 8 illustrates an existing named entity recognition result for an example input text. According to FIG. 8, it can be seen that seven entities 61 to 67 are recognized as a named entity recognition result. However, it can be seen that for a type of two entities 61 and 62 related to named entities, there is no information worthy to be extracted from the input text in terms of the task automation. On the other hand, it can be seen that there are expressions related to the meaning of the entities in the input text in most of other quantity expression (@NOH) type of entities among a type of entities related to numerical values.

For example, it can be seen that the meaning of 191.2 billion won 65, which is an entity related to money, is ‘assets’, the meaning of 182.5 billion won 66, which is an entity related to money, is ‘liabilities’, and the meaning of 64.2 billion won 67, which is an entity related to money, is a ‘deficit’. However, for 2022 64, which is an entity related to time (@TIM) among a type of entities related to numerical values, an expression related to the meaning of the entity may not be found in the input text.

Through analysis of multiple texts as illustrated in FIG. 8, a plurality of entity types for which selection of attributes is meaningful in terms of the task automation are presented in FIG. 10. That is, it may be understood entity types illustrated in FIG. 10 are ‘entities of a type of interest’, which are targets of attribute selection.

Together with a restriction on the entity types, a relation class may also be restricted to a meaningful relation class in order to find an attribute for the task automation. In some exemplary embodiments of the present disclosure, a relation class between an entity, which is a target of the attribute selection, and a candidate attribute functions as a kind of filter for the candidate attribute, and thus, there will be no need to classify relations of various classes like the existing relation extraction task. In consideration of this, in some exemplary embodiments of the present disclosure, the relation class between the entity and the candidate attribute may be determined as any one of relations of three classes: is-a, part-of, and no-relation. FIG. 9 is a diagram for describing the meaning of such three classes.

As illustrated in FIG. 10, the quantity type of entities may be further classified into a plurality of types 72 of entities in detail. For example, the quantity type of entities may include entity types of money (@MNY), percentage (@PNT), age (@AGE), amount (@AMT), count (@CNT), height (@HGH), length (@LEN), ordinal (@ORD), speed (@SPD), score (@SCR), weight (@WGH), temperature (@TMP), and the number of persons (@NOP). In FIG. 10, symbols 73 of each entity type are illustrated.

In some exemplary embodiments, a plurality of relation classes, which are classification targets for each entity type, may be different. For example, the plurality of relation classes, which are the classification targets for each entity type, may be at least one of a class: no-relation and two relation classes: is-a and part-of. In FIG. 10, a detection target relation class 74 and its expression 75 for each entity type are illustrated.

A process of selecting attribute of the entity (S400) will be described in more detail with reference to FIG. 5.

In the preprocessing process (S200), the input text may be segmented into the plurality of unit texts as described above. For convenience of understanding, a description will be provided on the assumption that the unit text is a sentence.

Selection of entities will be repeatedly performed sequentially for each sentence from a first sentence included in the input text (S401, S407, and S408).

In addition, since there is no need to perform an attribute identification operation in a sentence that does not include any entity of the type of interest, calculation amount consumption for selecting the entity may be minimized by skipping the sentence that does not include any entity of the type of interest (NO of S402).

The attribute identification operation will be performed on a sentence including one or more entities of the type of interest. There may be a sentence including a plurality of entities of the type of interest, and in this case, attribute identification will be performed for each entity of the type of interest (S403, S405, and S406).

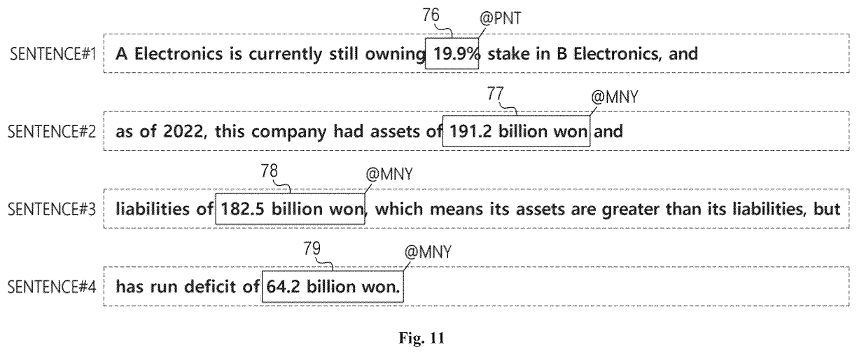

An attribute of a current entity may be selected among tokens of a current sentence (S404). The current sentence refers to a sentence that is being processed in the present turn, and the current entity refers to an entity that is being processed in the present turn. An operation related to attribute selection will be described in detail with reference to FIG. 11, which is a result of segmenting the input text in units of sentences through preprocessing of the example input text illustrated in FIG. 8 and then performing named entity recognition only on the entities of the type of interest illustrated in FIG. 10. FIG. 11 illustrates a situation where entities 76 to 79 of the type of interest are recognized one by one in SENTENCE 1 to SENTENCE 4 of the input text. When there is a sentence in which an entity of the type of interest is not recognized among SENTENCE 1 to SENTENCE 4, a process of performing an operation related to attribute selection on the sentence will be skipped.

As a preliminary task for selecting the attribute of the entity, morphological analysis may be performed on the input text. As a result of performing the morphological analysis, respective tokens included in the input text will be identified. Some of the tokens identified as described above may become a candidate attribute of the entity.

The candidate attribute may be selected among tokens other than tokens of parts of speech that do not include information, such as a postpositional particle, the ending of a word, a prefix, a suffix, a sign, and an adverb. Hereinafter, unless otherwise stated, the candidate attribute should be understood as the token other than the tokens of parts of speech that do not include information.

In addition, in some exemplary embodiments, the candidate attribute may be tokens positioned at a token distance within a reference value from a current entity. A token distance between a first token and a second token may be defined as (the number of tokens positioned between the first token and the second token)+1.

However, even though the token distance is long, there is a possibility that there is a close correlation between the tokens. In consideration of such a possibility, the candidate attribute may further include tokens whose context distances from the current entity satisfy a preset condition.

The context distance may be determined through dependency parsing, which is a known natural language processing task. In some exemplary embodiments, the tokens whose context distances from the current entity satisfy the preset condition may refer to tokens that have a dependency relation with the current entity as a result of the dependency parsing. Meanwhile, when the input text is a bullet-type expression or an expression in the form of a simple concatenation of nouns, the dependency parsing will be inaccurate. Therefore, in this case, the dependency parsing is not performed, and the candidate attribute may be determined based only on the token distance from the current entity.

In some exemplary embodiments, the candidate attribute may be limited to being in the same sentence as the current entity. That is, the attribute of the current entity may be selected among tokens included in a sentence including the current entity. Such exemplary embodiments may be adopted when the unit text is a sentence.

In some other exemplary embodiments, the candidate attribute may be selected among tokens that are in the same sentence as the current entity and are positioned at a token distance within a reference value from the current entity. For example, when an average sentence length included in the input text exceeds a reference length, the candidate attribute may be determined to be selected among the tokens that are in the same sentence as the current entity and are positioned at the token distance within the reference value from the current entity.

On the other hand, in some other exemplary embodiments, the candidate attribute may not be limited to being in the same sentence as the current entity. That is, the candidate attribute is not necessarily in the same sentence as the current entity, and a token included in a different sentence from the current entity may also become the candidate attribute as long as it is a token whose token distance from the current entity is within the reference value. Such exemplary embodiments may be adopted when the unit text is not the sentence. That is, such exemplary embodiments may be adopted when the unit text is a bullet-type expression or a noun enumeration-type expression. This is because it is difficult to regard the unit text comprising the bullet-type expression or the noun enumeration-type expression as having completeness in contents as much as a sentence.

In addition, in some exemplary embodiments, the candidate attribute may be selected among non-entity tokens that do not include the entity.

In some exemplary embodiments, the non-entity tokens may be limited to not including the type of interest and any type of entities recognized in existing named entity recognition. That is, the candidate attribute may exclude all types of entities that are recognizable. This is because it is likely that the entity will not describe the meaning of the code type of entity or the quantity type of entity regardless of its type.

However, in some other exemplary embodiments, an entity may be admitted as the non-entity token as long as is not an entity of the type of interest. It is likely that the entity will not describe the meaning of the code type of entity or the quantity type of entity regardless of its type, but it will not be regarded that there is never a case where an entity that is not the code type and the quantity type of entities most accurately describes the meaning of the code type or the quantity type of entity. Even in this case, when the attribute should be accurately identified, an entity other than the entity of the type of interest may be included in the non-entity token.

The candidate attribute may be required to be a non-entity token included in the same sentence as the current entity, a non-entity token positioned at a token distance within a reference value from the current entity, or a non-entity token included in the same sentence as the current entity and positioned at the token distance within the reference value from the current entity. Meanwhile, when the input text comprises a descriptive sentence, the candidate attribute may be required to be a non-entity token which is included in the same sentence as the current entity, a non-entity token which is positioned at a token distance within a reference value from the current entity or whose context distance satisfies a preset condition, or a non-entity token which is included in the same sentence as the current entity, which is positioned at the token distance within the reference value from the current entity, or whose context distance satisfies the preset condition.

In addition, the non-entity tokens may be limited to not including the type of interest and any type of entities recognized in the existing named entity recognition. Further, the non-entity token may include entities other than the entity of the type of interest.

In addition, in some exemplary embodiments, some of the candidate attributes may be excluded from the candidate attributes based on the relation class between the candidate attributes and the current entity. For example, a candidate attribute whose relation class with the current entity is a class: no-relation among the candidate attributes may be excluded from the candidate attributes.

Hereinabove, exemplary embodiments of various methods for selecting the candidate attribute have been described. By selecting the candidate attribute in various manners in consideration of various situations, it will become possible to accurately select the attribute.

### (1) Rule-Based Attribute Selection

An attribute of the current entity may be selected among a plurality of candidate attributes based on a rule. Hereinafter, it will be described in detail.

In some exemplary embodiments, a token distance between each of the plurality of candidate attributes and the current entity may be used in a process of selecting the attribute based on the rule. That is, the rule may be defined as assigning a higher score as the token distance between the candidate attribute and the current entity becomes shorter. However, the rule may ‘partially’ use the token distance. That is, the rule is not defined only by the token distance, and may include an additional input factor. The additional input factor may be a context distance. The additional input factor may be a relation class between each of the plurality of candidate attributes and the current entity.

In some exemplary embodiments, the relation class between each of the plurality of candidate attributes and the current entity may be used in the process of selecting the attribute based on the rule. That is, the rule may be defined as being satisfied when the relation class between the candidate attribute and the current entity has an attribute appropriate relation class.

For example, the attribute appropriate relation class may be a class: is-a regardless of a type of the current entity.

In addition, the attribute appropriate relation class may include both a class: is-a and a class: part-of regardless of the type of the current entity.

In addition, the attribute appropriate relation class may be a relation class determined based on the type of the current entity. For example, the attribute appropriate relation class may include at least one of the class: is-a and the class: part-of based on the type of the current entity. In a table in FIG. 10, relation classes 74, which are detection targets for each type 72 and 73 of entity are described, and it may be understood that a relation class other than no-relation among the relation classes 74, which are the detection targets, is the attribute appropriate relation class. For example, in a case of a relation class (@PNT) of a percentage type, the attribute appropriate relation class may include both the class: is-a and the class: part-of, while in a case of a relation class (@LEN) of a length type, the attribute appropriate relation class may include only the class: is-a. In other words, the attribute appropriate relation class may be different for each entity type.

Meanwhile, when the relation classes between the current entity and the plurality of candidate attributes are classified, a deep learning-based model may be used. In this case, since the attribute appropriate relation class is determined based on the type of the current entity, the relation classes between the current entity and the plurality of candidate attributes may be classified using a first relation extraction model when the type of the current entity is a first type, and the relation classes between the current entity and the plurality of candidate attributes may be classified using a second relation extraction model when the type of the current entity is a second type.

In this case, the first relation extraction model and the second relation extraction model may be models trained based on machine learning, receiving input data including a sentence, an entity, and an attribute, and outputting data related to what a relation between the entity and the attribute belongs to any one of a plurality of relation classes, the first relation extraction model may output data related to what the relation between the entity and the attribute belongs to any one of a plurality of first relation classes, the second relation extraction model may output data related to what the relation between the entity and the attribute belongs to any one of a plurality of second relation classes, and at least one of the plurality of first relation classes may include one or more first non-common relation classes which is not included in the plurality of second relation classes and at least one of the plurality of second relation classes may include one or more second non-common relation classes which is not included in the plurality of first relation classes.

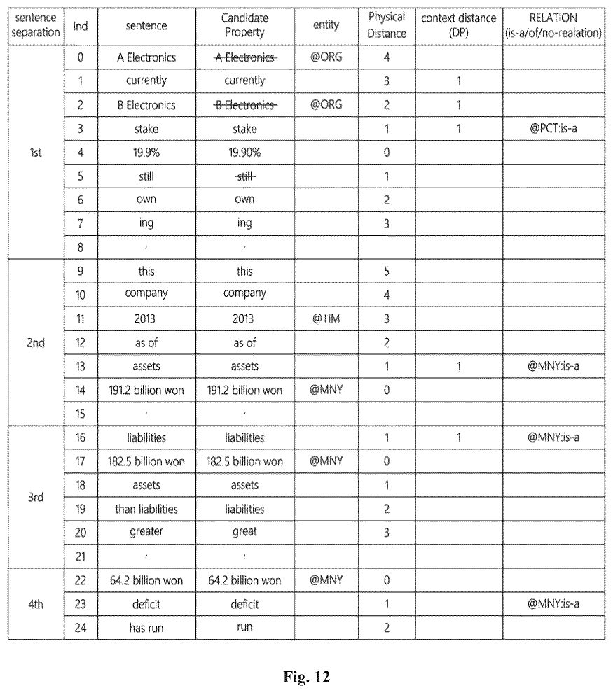

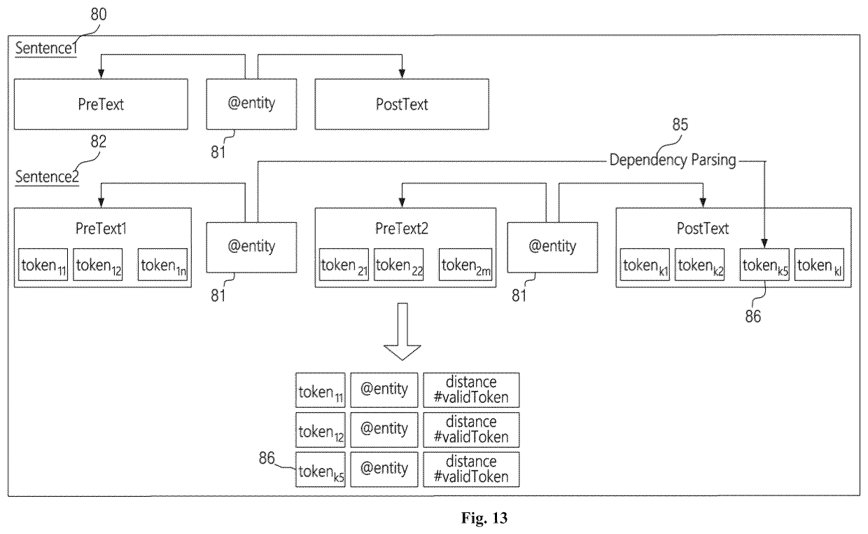

When the number of candidate attributes whose relation classes with the current entity have the attribute appropriate relation class is plural, an attribute of the current entity may be selected using at least one of a token distance and a context distance from each of the plurality of candidate attributes and the current entity. For example, for each of the plurality of candidate attributes, the rule may be defined so that the shorter the token distance from the current entity, the higher the score is evaluated and a candidate attribute whose context distance from the current entity satisfies a preset condition is selected as the attribute in a situation where the scores are the same as each other. FIG. 12 is a diagram for describing an attribute selection process according to such a rule.

FIG. 12 assumes a setting where tokens included in the same sentence as the current entity become candidate attributes and non-entity tokens are limited to not including any type of entities. In addition, it is to be noted that in a description of the relation class between each candidate attribute and the entity in FIG. 12, a relation class field is left blank in a case of no-relation or in a case where relation class classification is unnecessary, such that relation extraction is not performed. For example, in FIG. 12, “A electronics” and “B electronics” that are not non-entity tokens will be excluded from the candidate attributes, and accordingly, relation extraction with “19.9%” (@PCT), which is an entity of the type of interest, will not be performed.

An entity of the type of interest recognized in a first sentence will be “19.9%” (@PCT, index 4). “A electronics” and “B electronics” are also entities (@ORG), but it has been described several times in exemplary embodiments of the present disclosure that entities whose attributes are identified are limited to entities of a plurality of predetermined types of interest, and it has also been described that the plurality of types of interest include a type related to a code or a quantity. An entity type (@ORG) related to the organization name is not a type of interest in exemplary embodiments of the present disclosure.

In addition, since “A electronics” and “B electronics” are entities (@ORG) and “still” (index 5) is an adverb, “A electronics” and “B electronics” and “still” will be excluded from the candidate attributes. Accordingly, candidate attributes for identifying an attribute of “19.9%” (@PCT), which is the entity of the type of interest, will be “current” (index 1), “stake” (index 3), “own” (index 6), and “ing” (index 7). A candidate attribute having the class: is-a or the class: part-of as a relation class with “19.9%” among the candidate attributes is only “stake”, and thus, the candidate attribute “stake” will be selected as the attribute of “19.9%”.

Even assuming a situation where all candidate attributes have the class: is-a or the class: part-of as the relation class with “19.9%”, according to the rule defined so that the shorter the token distance from the current entity, the higher the score is evaluated and the candidate attribute whose context distance from the current entity satisfies the preset condition is selected as the attribute in the situation where the scores are the same as each other, “stack”, which is a candidate attribute whose token distance (physical distance) is shortest, will be selected as the attribute of “19.9%”.

An entity of a type of interest recognized in a second sentence will be “191.2 billion won” (@MNY, index 14). In the second sentence, “2012” (index 11) is also an entity (@TIM) and “,” (index 15) corresponds to a punctuation mark, and thus, candidate attributes for identifying an attribute of “191.2 billion won” (@MNY), which is the entity of the type of interest, will be “this” (index 9), “company” (index 10), “as of” (index 12), and “assets” (index 13). A candidate attribute having the class: is-a or the class: part-of as a relation class with “191.2 billion won” among the candidate attributes is only “assets”, and thus, the candidate attribute “assets” will be selected as the attribute of “191.2 billion won”.

An entity of a type of interest recognized in a third sentence will be “182.5 billion won” (@MNY, index 17). In the third sentence, “,” (index 21) corresponds to a punctuation mark, and thus, candidate attributes for identifying an attribute of “182.5 billion won”, which is the entity of the type of interest, will be “liabilities” (index 16), “assets” (index 18), “liabilities” (index 19), and “great” (index 20). A candidate attribute having the class: is-a or the class: part-of as a relation class with “182.5 billion won” among the candidate attributes is only “liabilities” (index 16), and thus, the candidate attribute “liabilities” (index 16) will be selected as the attribute of “182.5 billion won”.

An entity of a type of interest recognized in a fourth sentence will be “64.2 billion won” (@MNY, index 22). In the fourth sentence, candidate attributes for identifying an attribute of “64.2 billion won”, which is the entity of the type of interest, will be “deficit” (index 23) and “run” (index 24). A candidate attribute having the class: is-a or the class: part-of as a relation class with “64.2 billion won” among the candidate attributes is only “deficit” (index 23), and thus, the candidate attribute “deficit” (index 23) will be selected as the attribute of “64.2 billion won”.

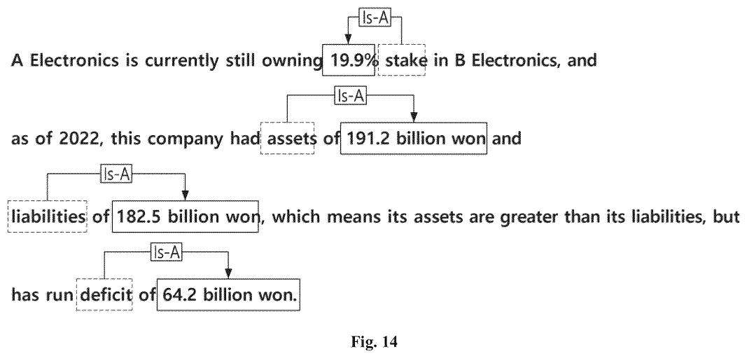

FIG. 13 is a diagram for describing, in more detail, an exemplary embodiment of a case where the token distance and the context distance between each of the plurality of candidate attributes and the current entity are reflected as input factors in the process of selecting the attribute based on the rule.

As illustrated in FIG. 13, the same entity 81 of a type of interest may be recognized together in two or more sentences 80 and 82. In this case, comparison between candidate attributes included in each sentence is necessary. For example, token distances between each candidate attribute and an entity 81 of the type of interest included in Sentence 1 80 and token distances between each candidate attribute and an entity 81 of the type of interest included in Sentence 2 82 may be compared with each other, and a candidate attribute having the shortest token distance may be selected as an attribute of the entity 81 of the type of interest. Meanwhile, when there are a plurality of candidate attributes having the shortest token distance, a candidate attribute whose context distance has a direct dependency relation among the plurality of candidate attributes may be selected as an attribute of the entity 81 of the type of interest. Token₁₁, token₁₂, and tokenₖ₅ 86 have the shortest token distance from the entity 81 of the type of interest, but only tokenₖ₅ 86 is decided to have a direct dependency relation with the entity 81 of the type of interest as a result of dependency parsing 85, and thus, tokenₖ₅ 86 may be selected as an attribute of the entity 81 of the type of interest.

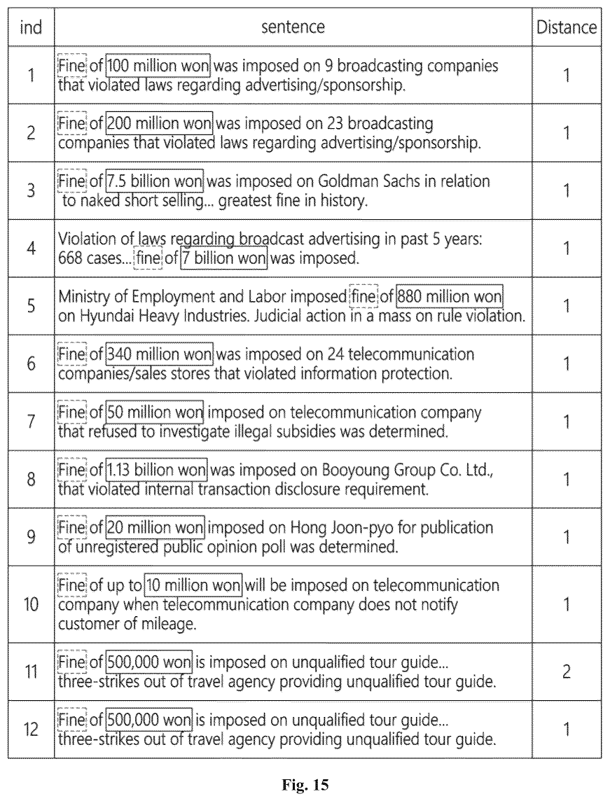

FIG. 14 is a diagram illustrating an attribute identification result described with reference to FIG. 12. In existing relation extraction, it was difficult to understand the meaning of the same type of several entities when the same type of several entities were recognized in the input text. That is, a money (@MNY) type of three entities are illustrated in FIG. 14, and it was difficult to understand whether, for example, 191.2 billion won was assets, liabilities, or a deficit, through a natural language processing technology. For this reason, it was difficult to implement task automation by processing an unstructured input text. However, in the above-described exemplary embodiments of the present disclosure, it is possible to accurately find the meaning of entities of a type of interest useful for task automation from the input text. Accordingly, a major obstacle to the task automation is removed. In addition, such an effect may be equally applied to statistical table-based attribute selection and deep learning-based attribute selection to be described later.

### (2) Statistical Table-Based Attribute Selection

In some other exemplary embodiments, an attribute of the current entity may be selected among a plurality of candidate attributes based on a statistics table. Hereinafter, it will be described in detail.

A statistical table may be generated by collecting records of performing entity-attribute identification on the input text using the above-described rule-based attribute selection method. In this case, representative values of the token distances decided at an identification point in time of entity-attribute and relation classes between the attributes and the entities may be recorded in the statistics table. The representative value may include at least one of an average value and a median value. In this case, a named entity itself of each entity does not have much meaning as statistical data. For example, in FIG. 15, 100 million won, which is a fine in Index 1 sentence, is different from 200 million won, which is a fine in Index 2 sentence, but such a difference in named entity will not be meaningful in performing attribute identification for each entity in exemplary embodiments of the present disclosure. Accordingly, the statistical data may include data such as a token distance representative value and the number of times of occurrence for each identification record of entity type-attribute.

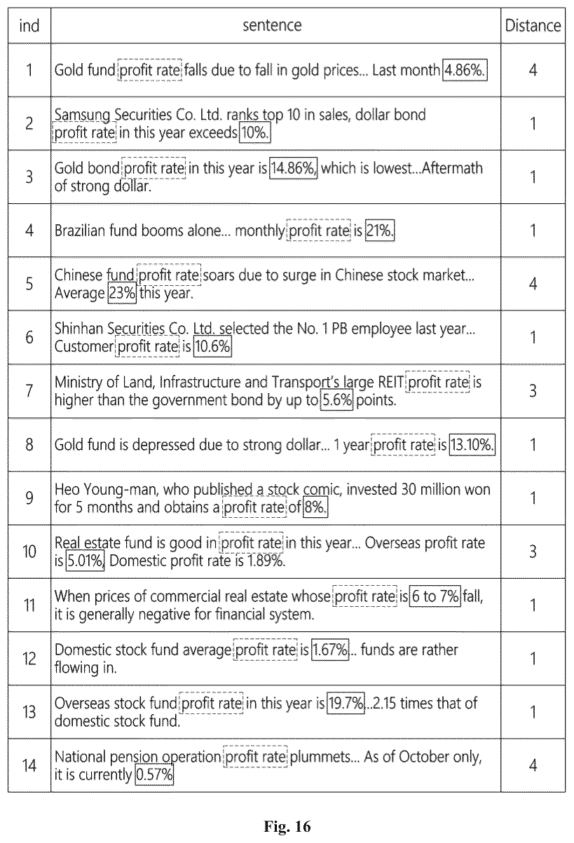

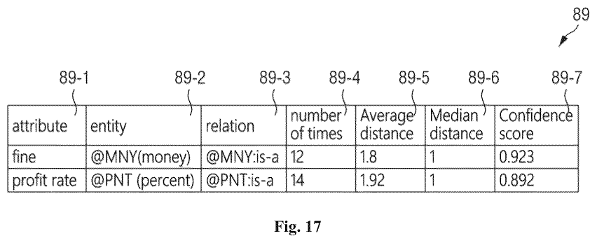

FIGS. 15 and 16 are records of performing entity-attribute identification, and FIG. 15 is a record when an entity type (@MNY) and a “fine” as its attribute were identified, and FIG. 16 is a record when an entity type (@PNT) and a “profit rate” as its attribute were identified. FIG. 17 is an example of a statistical table 89 in which the identification records of FIGS. 15 and 16 are collected. As illustrated in FIG. 17, the statistics table 89 may include an attribute 89-1, a type 89-2 of an entity, a relation class 89-3, the number of times of identification 89-4, and an average value 89-5 and a median value 89-6 of token distances, and may further include a confidence score 89-7.

The confidence score 89-7 may be calculated, for example, using Equation 1.

$$Confidence score = ( 1 - exp ( - alpha * frequency ) ) * exp ( - beta * distance ) \qquad [ Equation 1 ]$$

Here, frequency is the number of times of identification of corresponding entity type-attribute, distance is a representative value of a token distance between corresponding entity type and attribute, alpha is a weight of the frequency, and beta is a weight of the distance.

It may be understood that alpha and beta in Equation 1 are weights continuously adjusted based on attribute identification accuracy in a statistical table accumulation process.

A method for identifying an attribute of the current entity based on the statistics table will be described.

First, a record for each of a plurality of candidate attributes is retrieved from a pre-stored statistics table. In this case, for each candidate attribute, the record including matching between a token of the candidate attribute and a type of the current entity will be a retrieval target. When there is one candidate attribute for which a record has been retrieved among the plurality of candidate attributes, the candidate attribute will be selected the an attribute of the current entity.

When there are a plurality of candidate attributes for which records have been retrieved among the plurality of candidate attributes, the confidence score of each retrieved record may be adjusted using a difference between a representative value of a token distance and a relation class of the retrieved record and a token distance and a relation class between a first entity and a first candidate attribute on the input text. In this case, the greater the difference, the lower the confidence score of the retrieved record. Next, a candidate attribute having the highest value among the adjusted confidence scores of each retrieved record will be selected as the attribute. The present exemplary embodiment may be understood as a method for selecting the candidate attribute as the attribute when the candidate attribute shows a pattern similar to an existing statistical table for which attribute selection has been completed.

In addition, a confidence score according to Equation 1 may also be assigned to a new candidate attribute for which a record is not retrieved among the plurality of candidate attributes, the confidence score assigned as described above may be compared with the confidence score of the retrieved record, and the new candidate attribute for which the record is not retrieved from the statistical table may also be finally selected as the attribute according to a comparison result, a final selection result may be additionally generated, and accordingly, a record for the new candidate attribute may be included in the statistical table.

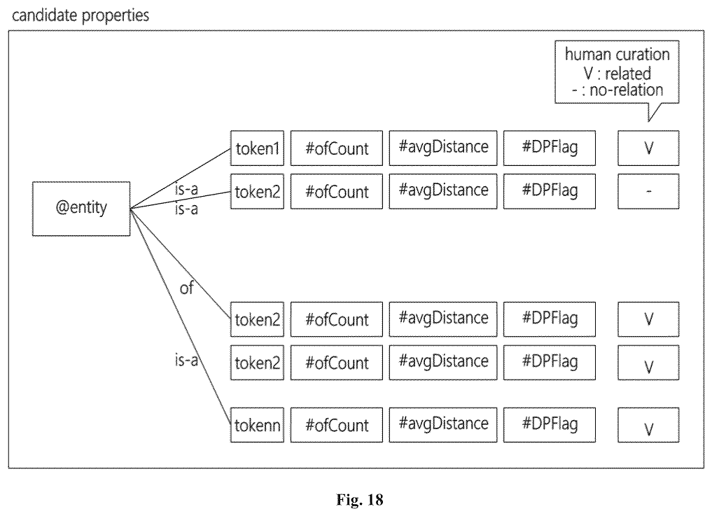

Meanwhile, the respective records recorded in the statistics table may be continuously cleansed through a human curation process. FIG. 18 illustrates an example screen that may be displayed in the human curation process. A human curator may delete inappropriate records of attribute selection records from the statistics table by performing the record cleansing through a screen as illustrated in FIG. 18.

### (3) Deep Learning Model-Based Attribute Selection

In some other exemplary embodiments, an attribute of the current entity may be selected among a plurality of candidate attributes using a deep learning-based attribute identification model. The attribute selection using the attribute identification model may be performed only when multiple training data are secured so that performance of the deep learning-based attribute identification model may be sufficiently increased. The training data may be automatically built using, for example, the statistical table described with reference to FIG. 17.

In some exemplary embodiments, training data for machine-learning the attribute identification model may include (target text, current entity, attribute, relation class between current entity and attribute, confidence score).

An example deep learning-based relation extraction method has been suggested in Hur et al. (2021). “K-EPIC: Named entity-PerceIved Context Representation in Korean Relation Extraction”, Appl. Sci. 2021, 11, 11472 (https://doi.org/10.3390/app112311472). According to Hur et al., the training data is (target text, subject entity, object entity, relation class between two entities), and the relation class between the two entities becomes a final output of a deep learning-based relation extraction model. In Hur et al., the relation extraction model calculates probability values for each of 30 relation classes and predicts a result according to input data as a relation class having the highest probability value.

Hur et al. suggest a deep learning-based model that outputs classification results for relation classes when (target text, subject entity, object entity) are input, while the attribute identification model according to some exemplary embodiments of the present disclosure may be machine-learned so as to receive input data of (target text, current entity, candidate attribute, relation class between current entity and candidate attribute) and output a relation class and a confidence score for the candidate attribute.

The attribute identification model may include an encoder outputting a second representation vector representing (target text, current entity, candidate attribute, relation class between current entity and candidate attribute) by concatenating or feature-fusing a relation vector representing the relation class between the current entity and the candidate attribute to a first representation vector of (target text, current entity, candidate attribute) and a full connected layer (FCL) receiving the second representation vector and outputting a confidence score.

In some exemplary embodiments, layers outputting the first representation vector included in the encoder of the attribute identification model may be obtained from a deep learning-based base model performing a relation extraction (RE) task between the entities, such as a representation vector extraction model suggested in Hur et al. That is, the attribute identification model may be generated through additional training using training data comprising (target text, entity, attribute, relation class between entity and attribute), based on the deep learning-based base model performing the relation extraction (RE) task between the entities.

As described above, the machine-learned attribute identification model may output the relation classes between the current entity and the candidate attributes included in the input data and the confidence score for each relation class. The confidence score for each relation class may include a confidence score of the class: is-a and a confidence score of the class: part-of.

So far, the method for identifying an attribute of an entity according to the present exemplary embodiment has been described with reference to FIGS. 4 to 19. It is to be noted that in the method for identifying an attribute of an entity described above, some operations may be added, changed, or excluded depending on a configuration of the input text related to the task automation, a type of entity of interest that is to be identified in the input text for the task automation, a task automation-related operation that is to be performed, or the like.

As an example, the method for identifying an attribute of an entity described above may include selecting an attribute of an entity included in the text among tokens that do not include the entity included in the text, outputting data on the selected attribute, and performing an RPA task using the entity and the attribute of the entity. The entity may be a code type of entity. The text may be an email body. In addition, the selecting the attribute of the entity may include determining relation classes between the respective candidate tokens and the entity and selecting the attribute of the entity among the respective candidate tokens using the relation classes of the respective candidate tokens. The relation class may be determined as one of two classes or one of three classes, and the candidate token may be some tokens selected based on an attribute of the token among the tokens that do not include the entity.

That is, in some exemplary embodiments of the present disclosure, it is possible to identify a code included in the email body, identify an attribute describing the exact meaning of the identified code in the email body, and provide an RPA service that performs a predefined action using (code, attribute).

The methods according to exemplary embodiments of the present disclosure described so far may be performed by executing a computer program implemented as computer-readable code. The computer program may be transmitted from a first computing device to a second computing device through a network such as the Internet, installed in the second computing device, and thus used in the second computing device. In addition, operations have been illustrated in a specific order in the drawings, but it is not to be understood that the operations should be necessarily performed in the specific order illustrated in the drawings or a sequential order or that all operations illustrated in the drawings should be performed in order to obtain a desired result. In a specific situation, multitasking and parallel processing may be advantageous.

### 3. System for Identifying Attribute of Entity

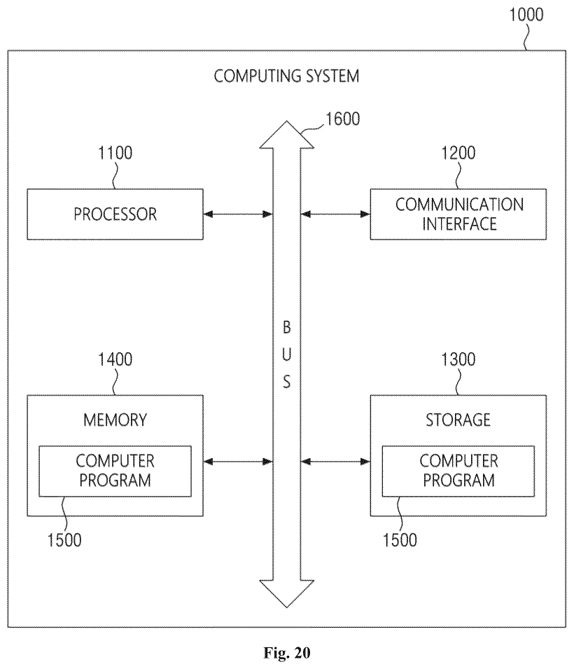

FIG. 20 is a block diagram illustrating a hardware configuration of a computing system according to some exemplary embodiments of the present disclosure. The computing system 1000 of FIG. 20 may include one or more processors 1100, a system bus 1600, a communication interface 1200, a memory 1400 loading a computer program 1500 executed by the processor 1100, and a storage 1300 storing the computer program 1500.

For example, the computing system 1000 of FIG. 20 may present a hardware structure of one or more computing systems constituting the system 100 for identifying an attribute of an entity described with reference to FIGS. 1 to 3.

The processor 1100 controls overall operations of respective components of the computing system 1000. The processor 1100 may perform an arithmetic operation on at least one application or program for executing methods/operations according to various exemplary embodiments of the present disclosure. The memory 1400 stores various data, commands, and/or information. The memory 1400 may load one or more computer programs 1500 from the storage 1300 in order to execute the methods/operations according to various exemplary embodiments of the present disclosure. The storage 1300 may non-temporarily store one or more computer programs 1500.

The computer program 1500 may include one or more instructions in which the methods/operations according to various exemplary embodiments of the present disclosure are implemented. When the computer program 1500 is loaded into the memory 1400, the processor 1100 may perform the methods/operations according to various exemplary embodiments of the present disclosure by executing the one or more instructions.

The computer program 1500 may include an instruction for recognizing one or more entities in an input text received through the communication interface 1200 or stored in the storage 1300 and an instruction for selecting an attribute of a first entity included in the one or more entities among tokens included in the input text. In addition, the instruction for selecting the attribute of the first entity may include an instruction for performing preprocessing on the input text, an instruction for performing segmentation of the preprocessed input text into a plurality of unit texts, an instruction for performing tokenization for each unit text, and an instruction for selecting the attribute of the first entity among tokens that do not include the recognized one or more entities.

In addition, the instruction for selecting the attribute of the first entity among the tokens that do not include the recognized one or more entities may include an instruction for selecting candidate attributes among the tokens that do not include the recognized one or more entities and an instruction for extracting a relation class between each candidate attribute and the first entity.

The instruction for extracting the relation class may use a relation class extraction model that classifies a relation class between a candidate attribute of the input data and the first entity into at least one of at least one class of a class: is-a and a class: part-of and a class: no-relation, as a machine learning-based relation class extraction model loaded into the memory 1400. In this case, as described above with reference to FIG. 10, an individual relation class extraction model may be used depending on a type of the entity, and a relation class extraction model may be loaded into the memory 1400 for each type of an entity of interest.

In addition, the instruction for selecting the attribute of the first entity may further include a method determination instruction for determining whether to perform a rule-based attribute selection method, to perform a statistical table-based attribute selection method, or to perform a deep learning model-based attribute selection method. To this end, the statistical table (not illustrated) as illustrated in FIG. 17 may be stored in the storage 1300 and then loaded into the memory 1400, and data of the attribute identification model described above may also be stored in the storage 1300 and then loaded into the memory 1400.

The statistics table may be updated according to the human curation procedure described with reference to FIG. 18, and in this case, an inappropriate attribute checked by the human curator may be removed from the records of the statistics table.

Although operations are shown in a specific order in the drawings, it should not be understood that desired results may be obtained when the operations must be performed in the specific order or sequential order or when all of the operations must be performed. The embodiments described above should be understood in all respects as illustrative and not restrictive. The scope of protection of the present invention should be interpreted in accordance with the claims below, and all technical ideas within the equivalent scope should be construed as being included in the scope of rights of the technical ideas defined by this disclosure.

What is claimed is:

1. A method for identifying an attribute of an entity, the method being performed by a computing system, the method comprising:
recognizing one or more entities in an input text; and
selecting an attribute of a first entity included in the one or more entities among tokens included in the input text,
wherein the selecting of the attribute of the first entity includes selecting the attribute of the first entity among tokens that do not include the recognized one or more entities.

2. The method of claim 1, wherein the recognizing of the one or more entities includes recognizing only any one type of entity of a plurality of predetermined types in the input text.

3. The method of claim 2, wherein the recognizing of only any one type of entity of the plurality of predetermined types includes recognizing a quantity type of entity or a code type of entity, and
the method further comprises performing a robotic process automation (RPA) task using the first entity and the attribute of the first entity.

4. The method of claim 2, wherein the recognizing of only any one type of entity of the plurality of predetermined types includes recognizing a quantity type of entity or a code type of entity, and
the method further comprises:
retrieving an input field corresponding to the attribute of the first entity; and
inputting the first entity as a value of the retrieved input field.

5. The method of claim 4, wherein the input text is a natural language text included in a medical record, and
the input field is included in one of a plurality of input forms belonging to an electronic medical record (EMR).

6. The method of claim 1, further comprising generating training data, the training data comprising entity-attribute pairs, each entity-attribute pair including a corresponding entity of the one or more entities and an attribute of the corresponding entity.

7. The method of claim 6, wherein the entity-attribute pair further includes a sentence including the corresponding entity, a type of the corresponding entity, and a relation class between the corresponding entity and the attribute.

8. The method of claim 1, wherein the selecting of the attribute of the first entity among the tokens that do not include the recognized one or more entities includes:
segmenting the input text into a plurality of unit texts; and
selecting the attribute of the first entity among tokens that is included in a unit text including the first entity and do not include the recognized one or more entities.

9. The method of claim 8, wherein the selecting of the attribute of the first entity among the tokens that is included in the unit text including the first entity and do not include the recognized one or more entities includes:
skipping the selecting of the attribute for a unit text in which any one type of named entity of a plurality of predetermined types is not recognized among the plurality of unit texts.

10. The method of claim 1, wherein the selecting of the attribute of the first entity includes:
selecting a plurality of candidate attributes among the tokens that do not include the recognized one or more entities, using a part of speech of each token;
determining a relation class between each of the plurality of candidate attributes and the first entity; and
selecting the attribute of the first entity using the determined relation class.

11. The method of claim 10, wherein the determining of the relation class includes determining the relation class as any one of three classes of relations: is-a, part-of, and no-relation.

12. The method of claim 10, wherein the determining of the relation class includes:
determining a plurality of relation classes corresponding to a type of the first entity; and
determining the relation class between each of the plurality of candidate attributes and the first entity as any one of the determined relation classes.

13. The method of claim 12, wherein the determining of the plurality of relation classes corresponding to the type of the first entity includes:
determining at least one of two relation classes: is-a and part-of as some of the plurality of relation classes corresponding to the type of the first entity; and
determining a class: no-relation as the other of the plurality of relation classes corresponding to the type of the first entity.

14. The method of claim 12, wherein the determining the relation class between each of the plurality of candidate attributes and the first entity includes selecting a candidate attribute having a relation class corresponding to a type of the entity as the attribute of the entity.

15. The method of claim 10, wherein the determining of the relation class includes:
determining the relation class between each of the plurality of candidate attributes and the first entity using a first relation extraction model when a type of the first entity is a first type; and
determining the relation class between each of the plurality of candidate attributes and the first entity using a second relation extraction model different from the first relation extraction model when the type of the first entity is a second type different from the first type,
the first relation extraction model and the second relation extraction model are models trained based on machine learning, receiving input data including a sentence, an entity, and an attribute, and outputting data related to what a relation between the entity and the attribute belongs to any one of a plurality of relation classes,
the first relation extraction model outputs data related to what the relation between the entity and the attribute belongs to any one of a plurality of first relation classes,
the second relation extraction model outputs data related to what the relation between the entity and the attribute belongs to any one of a plurality of second relation classes, and
at least one of the plurality of first relation classes includes one or more first non-common relation classes which is not included in the plurality of second relation classes and at least one of the plurality of second relation classes includes one or more second non-common relation classes which is not included in the plurality of first relation classes.

16. The method of claim 10, wherein the selecting of the plurality of candidate attributes includes excluding some of the plurality of candidate attributes from the plurality of candidate attributes using a relation between each of the plurality of candidate attributes and the entity, and
the selecting of the attribute of the first entity using the determined relation class includes:
determining a token distance between a candidate attribute and the entity for each of candidate attributes remaining after the excluding of some of the plurality of candidate attributes; and
selecting the attribute of the first entity among the candidate attributes remaining after the excluding of some of the plurality of candidate attributes, using the token distance of each of the candidate attributes.

17. The method of claim 16, wherein the selecting of the attribute of the first entity among the candidate attributes remaining after the excluding of some of the plurality of candidate attributes, using the token distance of each of the candidate attributes includes:
determining a context distance between the candidate attribute and the first entity; and
selecting the attribute of the first entity among the candidate attributes remaining after the excluding of some of the plurality of candidate attributes, using the token distance and the context distance of each of the candidate attributes.

18. The method of claim 17, wherein the determining of the context distance and the selecting of the attribute of the first entity among the candidate attributes remaining after the excluding of some of the plurality of candidate attributes, using the token distance and the context distance of each of the candidate attributes are performed only when the input text is a descriptive sentence.

19. The method of claim 1, wherein the selecting of the attribute of the first entity includes:
selecting a plurality of candidate attributes among the tokens that do not include the recognized one or more entities, using a part of speech of each token;
determining a token distance between each of the plurality of candidate attributes and the first entity; and
selecting the attribute of the first entity by partially using the token distance of each of the candidate attributes.

20. The method of claim 19, wherein the selecting of the attribute of the first entity further includes determining a relation class between each of the plurality of candidate attributes and the first entity, and
the selecting of the attribute of the first entity by partially using the token distance of each of the candidate attributes includes selecting the attribute of the first entity using the token distance of each of the candidate attributes and the determined relation class.

21. The method of claim 1, wherein the selecting of the attribute of the first entity includes:
selecting a plurality of candidate attributes among the tokens that do not include the recognized one or more entities, using a part of speech of each token;
retrieving a record for each of the plurality of candidate attributes from a pre-stored statistical table; and
selecting the attribute of the first entity using the retrieved record of each of the plurality of candidate attributes,
the pre-stored statistical table includes a record of each attribute,
the record includes a type of an entity, the number of times of extraction of the entity, information on a distance between an attribute and the entity, and a confidence score, and
the retrieved record is a record of a candidate attribute having a type of an entity coinciding with a type of the first entity.

22. The method of claim 21, wherein the selecting of the attribute of the first entity using the retrieved record of each of the plurality of candidate attributes includes:
calculating a difference between a record of the retrieved record of each of the plurality of candidate attributes and a distance between the first entity and a first candidate attribute on the input text;
adjusting the confidence score of the retrieved record for each of the plurality of candidate attributes using the calculated difference of each of the plurality of candidate attributes; and
selecting the attribute of the first entity using the adjusted confidence score of each of the plurality of candidate attributes.

23. The method of claim 21, wherein the record further includes information on a relation class between the attribute and the entity, and
the retrieved record is a record of the candidate attribute having a relation class value coinciding with a relation class between the first entity and the candidate attribute.

24. The method of claim 21, wherein the selecting of the attribute of the first entity using the retrieved record of each of the plurality of candidate attributes includes:
calculating a confidence score of a first candidate attribute using a token distance between the first candidate attribute and the first entity and a relation class between the first candidate attribute and the first entity when a record corresponding to the first candidate attribute of the plurality of candidate attributes is not retrieved from the pre-stored statistical table; and
selecting the attribute of the first entity by comparing the calculated confidence score with a confidence score of the retrieved record.

25. The method of claim 1, wherein the selecting of the attribute of the first entity among the tokens that do not include the recognized one or more entities includes:
selecting a plurality of candidate attributes among the tokens that do not include the recognized one or more entities, using a part of speech of each token;
constructing input data for each of the plurality of candidate attributes, the input data including a target text, the first entity, a candidate attribute, and a relation class between the first entity and the candidate attribute; and
inputting the input data for each of the plurality of candidate attributes into a pre-trained deep learning-based attribute identification model and selecting the attribute of the first entity among the plurality of candidate attributes using data output from the pre-trained deep learning-based attribute identification model.

26. The method of claim 25, wherein the pre-trained deep learning-based attribute identification model is generated through additional training using training data comprising the target text, an entity, an attribute, a relation class between the entity and the attribute, based on a deep learning-based base model performing a relation extraction (RE) task between the entities.

27. A system for identifying an attribute of an entity, comprising:
a storage;
a communication interface;
a memory configured to load a computer program; and
one or more processors configured to execute the computer program,
wherein the computer program includes:
an instruction configured to cause the one or more processors to recognize one or more entities in an input text received through the communication interface or stored in the storage; and
an instruction configured to cause the one or more processors to select an attribute of a first entity included in the one or more entities among tokens included in the input text, and
the instruction configured to cause the one or more processors to select the attribute of the first entity includes an instruction configured to cause the one or more processors to select the attribute of the first entity among tokens that do not include the recognized one or more entities.
